(19) United States
(12) Patent Application Publication
(10) Pub. No.: US 2010/0144854 A1
(43) Pub. Date: Jun. 10, 2010

(54) USE OF RHODAMINE 6G AS A MEDICINAL AGENT FOR TREATING MALIGNANT NEOPLASMS AND AMILOIDOSES

(76) Inventor: Mikhail Kutushov, Moscow (RU)

Correspondence Address: KEVIN D. MCCARTHY;ROACH BROWN MCCARTHY & GRUBER, P.C., 424 MAIN STREET, 1920 LIBERTY BUILDING, BUFFALO, NY, 14202, US

(21) Appl. No.: 12/531,413

(22) PCT Filed: Mar. 16, 2007
(86) PCT No.: PCT/RU07/00130

§ 371 Date: Feb. 11, 2010

(51) Int. Cl. A61K 31/352; A61P 35/00

(52) U.S. Cl. 514/454

(57) ABSTRACT

Rhodamine 6G is used as a medical agent for treating malignant neoplasms and amyloidoses at a dosage ranging from 1 mg to 10 g in the form of an aqueous, physiological, or alcoholic solution. For different purposes, the rhodamine G6 solution is orally administered before or after meals, or rhodamine G6 solution is rectally administered or the rhodamine G6 1% solution is intravenously injected, or the rhodamine G6 is administered in the form if a suppository or a 1-5% ointment.

### FIELD OF THE INVENTION

The present invention relates to medicine, and in particular to medicinal preparations used for treating amyloidoses and oncological diseases.

### BACKGROUND OF THE INVENTION

The medicinal preparation “fluoracil” is known, whose antitumor activity is determined by its conversion in cancer cells into a competing inhibitor for an enzyme involved in the synthesis of nucleic acids. This preparation is used in the form of intravenous injections to treat inoperable and recurring cancer of the stomach, large intestine and rectum, breast cancer, ovarian cancer and also pancreatic cancer, although the preparation is highly toxic and its use can cause side effects, such as suppression of hematosis, diarrhea, loss of appetite, vomiting and ulcerative stomatitis. Furthermore, this preparation is contraindicated in patients whose condition is serious or who are suffering from gastric ulcers and duodenal ulcers, manifested as functional liver failure. The preparation melphalan is known, which is used to treat amyloidoses. In this connection melphalan is used only on an intermittent basis to treat amyloidoses since it is a toxic preparation and possible side effects of its use are the development of a secondary tumor (acute leukemia) or myelodysplasia syndrome. A milder regimen is to use melphalan every 4-6 weeks, in courses of treatment over 7 days in combination with prednisolone. The treatment is long-term, for not less than one year. The treatment outcomes are worse in patients suffering from chronic cardiac insufficiency (CCI) but are noticeably better in patients with nephrotic syndrome. Thus, in patients who respond to treatment the life extension can be 5 years. The preparation of colchicine is also known for treating amyloidoses. However, the preparation has very pronounced side effects, and does not have a satisfactory curative effect. Doxorubicin, antibiotic having an antitumor action, is highly toxic. Closest to the claimed agent as regards objective and action is a medicinal agent having an antitumor and immunomodulatory action, characterized in that it contains pharmacologically acceptable derivatives of tri-n-aminotriphenylchloromethane, for example its tetramethyl, pentamethyl and hexamethyl derivatives (RU No. 2257200, 2004). The disadvantages of this known medicinal agent are its short action time, low effectiveness in the treatment of a number of tumors, low effectiveness in the treatment of amyloidoses, and its relative toxicity.

The use of rhodamine 6G as a red dye and colorant is known, which is used to dye skin and leather, paper, soap, as an analytical reagent in the determination of a number of metals, and also in medicine and biology as a staining dye in microscopy. The use of rhodamine as an auxiliary component of a composition for disinfecting seeds is known (RU No. 2282961, 2006). The object of the present invention is to provide an accessible and inexpensive medicinal agent having a broad spectrum of curative action. The technical result enabling this object to be achieved consists in the formulation of a medicinal agent with a broad range of application in oncological diseases and amyloidoses, with reduced toxicity and few side effects. The essence of the invention consists in the use of rhodamine 6G in the form of 9-(2-ethoxycarbonyl)phenyl)-3,6-bis(ethylamino)2,7-dimethylxanthyl chloride of the chemical formula C₂₈H₃₁N₂O₃.Cl, as an agent for treating malignant neoplasms and amyloidoses in a dosage of the agent ranging from 1 mg to 10 g.

As an agent for treating malignant neoplasms and amyloidoses rhodamine 6G can be used in the form of an aqueous, physiological or alcoholic solution. For different purposes a solution of rhodamine 6G is administered orally before or after meals, or a rhodamine 6G is administered rectally, or a 1% rhodamine 6G is administered intravenously, or rhodamine 6G is used rectally in the form of a suppository, or rhodamine 6G is used in the form of a 1-5% ointment. A medicinal preparation based on rhodamine 6G can be used orally in the form of solutions, a powder taken before or after meals, per rectum in the form of solutions or suppositories, externally or topically in the form of suspensions and ointments, intravenous and cavity infusions. When used in a wide range of dosages it does not produce any allergic reactions or other side effects, and its effectiveness (in the treatment of appropriate diseases) is greater than, for example, preparations selected from the prior art.

### DETAILED DESCRIPTION OF THE PREFERRED EMBODIMENTS

Rhodamine 6G with the chemical formula C₂₈H₃₁N₂O₃.Cl was used as the medicinal agent to treat malignant neoplasms and amyloidoses at a dosage ranging from 1 mg to 10 g, in the form of an aqueous, physiological or alcoholic solution. For different purposes a rhodamine 6G solution was administered orally before or after meals, or a rhodamine 6G solution was administered rectally, or a 1% solution of rhodamine 6G was administered intravenously, or rhodamine 6G was administered rectally in the form of suppositories, or rhodamine 6G was used in the form of a 1-5% ointment.

In one aspect of the invention, rhodamine 6G is administered to a subject to reduce a chance of developing in said subject a malignant neoplasm, comprising a dosage ranging from 1 mg to 10 g.

The results of the use of rhodamine 6G for therapeutic purposes are shown in the Examples.

### Example No. 1

Patient V. Age 73. Diagnosis: amyloidosis. The patient felt lethargic and “under the weather”. A CT brain scan showed degenerative changes and foci typical of amyloidosis. Treatment was prescribed, including administration of a medicinal preparation based on rhodamine 6G. The first course of treatment with the preparation consisted of 15 drops of a 1% solution in a glass of water three times a day by mouth, at the evening meal in addition a microenema of 10 drops of a 1% solution in 30 ml of water, and at night a 1% suppository containing the preparation. During the first week there was basically no change in the patient's condition, though the patient noted that for several hours after administration of the treatment he felt less drowsy. By the third week after the start of treatment the patient's condition had improved. In the words of relatives, the patient was “feeling better”. Three months after the start of treatment with the preparation a CT scan showed a reduction of amyloid foci in the brain. One year after treatment the patient's condition had significantly improved, and he felt better and the amnesia had to some extent improved. Blood analyses against the treatment background were normal.

### Example No. 2

Patient L, age 43.

Three years previously the left kidney was removed and prophylactic chemotherapy was carried out to treat renal cell carcinoma. A CT projection scan of the left kidney revealed a tumor-like mass of size 3.5×5.4×3.7 cm, and also four tumor-like round formations of diameter 2.0-2.3 cm in the left lung. Severe dyspnea, diagnosis adynamia. Treatment was prescribed, including the use of a medicinal preparation having an anti-tumor action. The preparation rhodamine 6G was administered by mouth in the form of a drink containing 12 drops of a 1% solution three times a day, and in the form of a microenema. After 4 weeks the dyspnea and pain had decreased. Blood analyses against the treatment background normalised over 3 months. A repeat course of treatment with the preparation was then prescribed, which was administered rectally in the form of a 1% suppository and also by inhalation for 6 months. Over the course of the treatment the tumor in the right kidney and the metastases in the lungs disappeared. The patient regained weight.

### Example No. 3

Patient T. Age 54. Diagnosis: adenocarcinoma of the right ovary. Metastases in the liver. A hysterectomy had been carried out 4 years before. There was a recurrence of the cancer after a year. The patient complained of pain in the pelvis and difficulty in urination. A CT scan of the region between the bladder and the rectum showed a tumor-like formation 4×5×4 cm in size. The patient's condition was moderately severe; pale skin. Treatment began with the preparation rhodamine 6G in the form of 20 drops of a 1% solution in half a glass of water by mouth, and also the form of a microenema. The course of treatment lasted 3 months. During the treatment the tumor decreased in size to 1.2×0.9×0.3 cm. In the second week after the start of treatment the stools became normal and the feeling of pain disappeared. Blood analyses against the treatment background became normal over 2 months. A full course of treatment with the preparation was carried out over 6 months. During the course of the treatment the tumor in the region of the bladder and rectum disappeared. The patient regained weight.

### Example No. 4

Patient G. Age 43. Diagnosis: Malignant glioblastoma of the left brain. Recurrence of cancer. Two years previously a tumor had been removed in connection with a brain glioblastoma. The patient complained of headaches, nausea and vomiting. Projection NMRT (nuclear magnetic resonance tomography) of the right operation scar in the brain substance revealed a tumor with irregular edges of size 2.8×2.9 cm. Patient's condition moderately severe; pale skin. The patient was prescribed the preparation rhodamine 6G in a concentration of 500 mg per 500 ml of sterile physiological saline solution intravenously, as drops. The procedure took 40 minutes. Subsequent procedures were carried out in exactly the same sequence twice a week. The course of treatment lasted 3 months. During the treatment the tumor reduced in size to 0.3×0.4 cm. After two infusions of the preparation the symptoms of nausea and pain had decreased. The preparation was administered over 6 months as a single dose once every 2 weeks. After 9 months' treatment no tumor could be detected on the CT scan.

### Example No. 5

Patient L. Age 64. Diagnosis: Primary cancer of the liver. One and a half years before, resection of part of the right hepatic lobe on account of liver cell carcinoma. Patient complained of pain in the right hypochondrium, dyspnea, ascites, intermittent retention of stools. Patient's condition moderately severe, loss of appetite, jaundice. An ultrasound scan revealed a tumor in the left lobe of the liver with irregular edges of area 4×5 cm. The patient underwent bougienation of the umbilical vein. 2 g of sterile rhodamine 6G preparation dissolved in 100 ml of physiological saline were administered through a catheter twice a day over 2 weeks. Subsequent procedures were carried out in exactly the same way twice a week. Only the preparation was administered intravenously. The course of treatment lasted 3 months. During the treatment the tumor in the affected region decreased in size to 0.5×0.7 cm. After 4 months a connective tissue scar of size 0.2×0.3 cm remained at the site of the tumor in the liver. During the treatment with the preparation there was a positive change in the blood analyses.

Results of full blood analyses

| Main indices | | 12 Nov. 2004 | 19 Jan. 2005 | 14 Feb. 2005 | 17 Apr. 2006 |
|---|---|---|---|---|---|
| Erythrocytes | | 3.22 | 3.60 | 4.37 | 4.67 |
| Hemoglobin | | 74 | 80 | 117 | 123 |
| Thrombocytes | | 315 | 362 | 286 | 310 |
| Leukocytes | | 7.2 | 7.8 | 5.8 | 6.7 |
| Basophils | 0-1% | 1 | — | — | 1 |
| Eosinophils | 0-5 | 1 | 2 | 2 | 1 |
| Nuclear rods | 1-6% | 1 | 1 | — | — |
| Nuclear segments | 45-70 | 21 | 44 | 63 | 65 |
| Lymphocytes | 18-40% | 9 | 16 | 23 | 37 |
| Monocytes | 2-9% | 15 | 4.3 | 4 | 9 |
| ESR 1-15 mm/hr | | 3.5 | | | 8.5 |

Results of biochemical blood analyses

| | Content in | | | | | |
|---|---|---|---|---|---|---|
| Main indices | normal range | | 10 Nov. 2004 | 20 Dec. 2005 | 04 Feb. 2005 | 07 Apr. 2005 |
| Total protein | 65.0-85.0 | | 43.43 | 49.90 | 78 | 64.34 |
| Creatinine | gl | | 98.0 | 83 | 79 | 67 |
| Cholesterol | 1.66-8.3 | | 6.21 | 5.62 | 5.75 | 7.03 |
| Total bilirubin | 2.90-6.54 | | 11.0 | 24.2 | 8.4 | 8.0 |
| Bilirubin | 8.5-20.5 | | 3.0 | 22.3 | 3.3 | 2.0 |
| direct | up to 25% | | 8.0 | 11.9 | 5.1 | 6.0 |
| Bilirubin | up to 25% | | 82 | 96 | 84 | 90 |
| indirect | up to 100 | el | 107.2 | 102 | 89 | 78.0 |
| Amylase | 95-110.0 | | 114.0 | 140 | 102.8 | 110.0 |
| Chlorine | 135.0- | | 114.20 | 115.3 | 114.31 | 134.2 |
| Potassium | 3.5-6.0 | | 2.72 | 2.32 | 2.28 | 2.15 |
| Serum iron | 8.8-27.0 | | 190.4 | 224.7 | 94.4 | 43.7 |
| AST | 5-40 | el | 554.5 | 432.9 | 103.8 | 30.4 |
| ALT | 5-40 | el | 665.8 | 426 | 154.2 | 40 |
| Phosphatase, alk. | up to 104 | el | 5.71 | 5.48 | 4.55 | 6.7 |
| LDG | 225-450 | el | 879 | 556 | 332 | 362 |

At the present time the patient has no complaints.

### Example No. 6

Patient N. Age 55. Diagnosis: adenocarcinoma of the large intestine. Metastases in the liver. Ascites. Three years previously a resection of the large intestine was performed with insertion of an anastomosis end to end. A full course of polychemo-therapy was given. Two years after the treatment the patient complained of pain in the bottom of the abdomen and tenesmus. The patient presented complaining of acute weakness, constant pain in the abdomen, coughing, dyspnea, ascites, retention of stools. Patient's condition serious, loss of appetite, pale cyanosed skin, abdomen with signs of ascites. Blood analyses revealed reduced erythrocyte, hemoglobin, leukocyte and albumin counts, and an increase in ALT and AST. A lump of irregular shape 2×4 cm in size with irregular edges was detected by computerized tomography in the right renal lobe. A large lump 4×5.4 cm in size and free liquid in the peritoneal cavity were also detected. The patient was prescribed the preparation rhodamine 6G in the form of intravenous infusions, microenemas and liquid. The infusion solution consisted of 200 mg of sterile rhodamine 6G dissolved in 500 ml of physiological saline. The infusions were administered weekly over the course of 3 months. The enemas comprised a solution of 20 g of rhodamine 6G in 1500 ml of boiled water. The enemas were administered three times a week over the course of 2 months. In order to reinforce the treatment the preparation was administered orally in the form of a 1% solution of rhodamine 6G in 200 ml of boiled water over 20 minutes before meals, three times a day. Ten days after the start of treatment the feeling of pain had completely disappeared and the patient's appetite returned. In the third week of treatment the dyspnea was less severe. In the course of treatment the tumor in the region of the anastomosis decreased in size to 0.2×0.3 cm. After 3 months the treatment was discontinued for 1 month, and was then resumed for a further 2 months. Over the course of the treatment no metastases were detected in the liver and no tumor was detected in the region of the anastomosis. A marked step-wise improvement in the blood indices was observed, directly associated with the treatment with the preparation.

Results of blood analyses

| Main indices | | 9 Oct. 2004 | 24 Nov. 2004 | 09 Dec. 2004 | 09 Jan. 2005 |
|---|---|---|---|---|---|
| Erythrocytes | | 3.20 | 3.84 | 4.44 | 4.87 |
| Hemoglobin | | 74 | 90 | 115 | 128 |
| Thrombocytes | | 120 | 210 | 326 | 440 |
| Leukocytes | | 9.2 | 12.0 | 5.2 | 5.3 |
| Basophils | 0-1% | 1 | — | — | 1 |
| Eosinophils | 0-5 | 1 | 2 | 2 | 1 |
| Nuclear rods | 1-6% | 0 | 1 | — | 2 |
| Nuclear segments | 45-70 | 2 | 6 | 6 | 5 |
| Lymphocytes | 18- | 35 | 49 | 58 | 69 |
| 40% | | 13 | 4 | 7 | 9 |
| Monocytes | 2-9% | 36 | 12 | 10 | 12 |
| ESR 1-15 nm/hr | | | | | |

Results of biochemical blood analyses

| | Content in | | | | | |
|---|---|---|---|---|---|---|
| Main indices | normal range | | 09 Oct. 2004 | 24. Nov. 2004 | 09 Dec. 2004 | 09 Jan. 2005 |
| Total protein | 65.0-80 | g/l | 51.23 | 62.26 | 65 | 82.53 |
| Creatinine | 85.0 | g/l | 118.0 | 89 | 74 | 69 |
| Cholesterol | 1.66-8.3 | | 6.21 | 5.62 | 5.75 | 7.03 |
| Total bilirubin | 2.90-6.54 | | 11.0 | 24.2 | 8.4 | 8.0 |
| Bilirubin | 8.5-20.5 | | 3.0 | 22.3 | 3.3 | 2.0 |
| direct | to 25% | | 8.0 | 11.9 | 5.1 | 6.0 |
| Bilirubin | to 25% | | 82 | 96 | 84 | 90 |
| indirect | to 100 | el | 107.2 | 102 | 89 | 78.0 |
| Amylase | 95-110.0 | | 114.0 | 140 | 102.8 | 110.0 |
| Chlorine | 135.0- | | 114.20 | 115.3 | 114.31 | 134.2 |
| Potassium | 3.5-6.0 | | 2.72 | 2.32 | 2.28 | 2.15 |
| Serum iron | 8.8-27.0 | | 190.4 | 104.7 | 54.8 | 42.9 |
| AST | 5-40 | el | 265.7 | 125.8 | 45.7 | 29.5 |
| ALT | 5-40 | el | 465.8 | 326 | 69.2 | 79 |
| | to 104 | el | 623 | 332 | 242 | 228 |
| | 225-450 | el | | | | |

Weight regained. Patient complained of no symptoms in 2 years.

### Example No. 7

Patient Sh. Age 55.

The patient presented complaining of a sharp pain when urinating and of retention and reddish color of the urine. On examination: Ultrasound scan—prostate approximately 85 cm³, tumor with irregular contours, 34×40 mm² in size. PSA-49.9 mg/ml.

Needle biopsy—adenocarcinoma of the prostate.

Diagnosis: Adenocarcinoma of the prostate. The patient refused surgical intervention and chemotherapy.

Treatment with the medicinal preparation rhodamine 6G was carried out.

The patient received the preparation rhodamine 6G for 40 days in the form of a powder in an amount of 300 mg twice a day during mealtimes, and further rhodamine 6G in an amount of 200 mg 30 min, after meals. At night a suppository containing 2% of the preparation was administered. During the treatment period the pain experienced on urination decreased in intensity, the color of the urine became normal, and according to an ultrasound investigation the size of the prostate decreased by 40%, the tumor being sharply defined, of size 13×14 mm². Treatment was then continued by administering rhodamine 6G for 2 weeks in an amount of 300 mg twice a day at mealtimes, and correspondingly in an amount of 300 mg of rhodamine 6G, and in addition treatment enemas were prescribed daily before sleeping, in an amount of 2.0 g of rhodamine 6G in 50 ml of boiled water.

During the subsequent examination, in the words of the patient “the symptoms of the disease (sharp pain on urination, retention of urine, etc.) have finally disappeared”. Ultrasound check showed prostate approximately 35 cm³ in size, and tumor had decreased in size to 3×4 mm², PSA—1.0 mg/ml.

No recurrence of the cancer was observed over 2 years.

### Example No. 8

Patient T, age 52.

In connection with the detection of carcinoma of the left ovary the patient underwent a hysterectomy.

After the operation the patient was given a 2-year course of chemotherapy. The patient subsequently complained of pain in the rectum and tenesmus.

A CT scan of the region between the rectum and the bladder revealed a tumor 13.0×12.5 cm² in size, which had penetrated through the walls.

Diagnosis: Recurring cancer of the right ovary.

In view of the ineffectiveness of continuing the chemotherapy, the patient was treated with the preparation in the form of an enema. Initially rhodamine 6G was administered in a dose of 5 g per day for 21 days.

On the third day after the start of treatment the severity of the pain decreased, and the urge to urinate and defecate became less frequent.

Ultrasound scan: After 2 weeks the tumour was 3.2×3.4 cm² in size, and after a further 4 weeks was 1.2×1.23 cm² in size; a sharp boundary between the tumor and walls of the rectum and bladder was observed.

An ultrasound check after 3 months revealed a round lump of size 0.3×0.4 cm² with clearly defined boundaries.

On further examination the patient's condition was found to be satisfactory, and an ultrasound scan revealed a lump of size 0.4×0.3 cm² with sharp contours. At the present time the patient is healthy.

### Example No. 9

Patient B, age 54. The patient had undergone a partial gastrectomy. Histological conclusion: Tubular, moderately differentiated adenocarcinoma infiltrating the stomach wall as far as the muscular layer. The operation was unsuccessful, and only herbs (hemlock, aconite, etc.) were taken, followed by three periods without solid food for 5, 7 and 7 days. CT result: No space-occupying lesions or localized and infiltrating changes were detected in the pulmonary tissue; localized area of fibrosis in basal sections S9 of lower lobe of left lung. Clear contours of bronchi, order 1-3, no liquid detected in the pleural cavities. Organs of the abdominal cavity: liver normal shape and position; smooth sharp contours, structure of parenchyma uniform, density unchanged. In right lobe of the liver lump with irregular contours, 4×5 cm in size. Intrahepatic and extrahepatic bile ducts not enlarged; gall bladder normal size. Free liquid in abdominal cavity. Results: Total blood analysis: Hemoglobin—86, leukocytes—47, lymphocytes 48. Remaining indices in normal range.

The tumor and metastases continue to grow. Treatment with rhodamine 6G was suggested, 10 g dissolved in 250 ml of water three times a day over 20 minutes before meals. 5% rectal suppositories at night. Treatment according to this regimen was continued for 3 months.

No primary tumor detected on a CT scan after 4 months; metastases of small size (1×2 cm) in the right lobe of the liver, with signs of calcification. Results of total blood analysis of 23 Dec. 2006:

Hemoglobin—134, leukocytes—4000, lymphocytes 43. Remaining indices in normal range.

No complaints at the present time. Weight 76 kg.

### Example No. 10

Patient T, age 67. Diagnosis: Ischemic heart disease: Atherosclerosis, cardiosclerosis, atrial fibrillation. Amyloidosis? On account of deterioration in the patient's condition (increased frequency of dyspnea) the patient was hospitalised. On admission the patient's overall condition was fairly serious. Skin and visible mucous membranes normal colour, moderately cyanosed lips. Heart sounds—muffled, arrhythmia—atrial fibrillation with frequency of ventricular contractions 110 beats/min (pulse deficiency 12), blood pressure 100/60 mm Hg. In left lung, dull percussion sound below the middle of the shoulder blade; no breathing detected above this region. Individual moist rales heard to the right in the projection of the lower lobe. Diseased liver, lower edge extending 4 cm below the level of the costal margin. Edema of the legs as far as middle third of femur and anasarca were diagnosed.

Low voltage waves recorded on ECG, taxistological shape of atrial fibrillation, QS in leads V1-V4. An echocardiogram showed the following: dilation of the left (front-rear size 50 mm) and right atrium (56×52 mm); pseudohypertrophy of the myocardium of the ventricular septum (thickness in diastole 17 mm) and rear wall of the left ventricle (thickness in diastole 15 mm); normal size of left and right ventricle (end-diastolic sizes 49 mm and 24 mm respectively). Disturbed diastolic relaxation of myocardium of left ventricle was noted. Total blood analysis showed: Hypochromic anemia (erythrocytes 2.66−2.77×1012/1, hemoglobin 95 g/l); moderate leukocytosis (10.3), leukocyte shape unchanged, quicker ESR (43 mm/hr). In the biochemical blood analysis the level of total protein was reduced (down to 50 g/l), while the levels of urea (to 9.66 mmol/l) and creatinine (to 149 mmol/g) were both raised. Proteinuria was observed in the urine (0.05 g/l). Ultrasound investigation of organs of the abdominal cavity: enlarged liver (right lobe—16.9 cm), echogenicity reduced, and irregular structure; portal vein−15 mm. Gall bladder: 52×22 mm, wall up to 4 mm, unobstructed lumen. Pancreas: uniform, echogenicity raised, focalized diffuse changes. Right kidney—102×51 mm, left kidney—1023×54 mm, cortical layer of both kidneys—1.8 mm. Calyculate-pelvic system of both kidneys not enlarged. Fogging of the lung pattern and diffuse changes were observed on an anterioposterior chest X-ray. A needle liver biopsy was performed in order to exclude amyloidosis. Signs of primary amyloidosis were detected. On the basis of these data the following diagnosis was made: primary generalized amyloidosis with pronounced damage to the heart, kidneys and liver. Conventional treatment did not produce any improvement and the patient's condition progressively worsened. It was decided to carry out treatment with the preparation rhodamine 6G, in a dose of 5 g dissolved in 250 ml of water three times a day over 20 minutes before meals. 5% suppositories in the rectum at night. Treatment according to this regimen was continued for 2 months. The condition of the patient gradually improved, although in order to accelerate the elimination of amyloids intravenous administration of the preparation in a dose of 2 g was prescribed. Intravenous administration twice a week. The course of treatment lasted 2 months. On the echocardiogram the size of the heart was found to be reduced as a result of the treatment. On the EKG the voltage waves were in the normal range, and there was no fibrillation. On the echocardiogram: dilation of the left and right atria was significantly reduced. The liver and kidneys were normal size on an ultrasound scan. Blood and urine analyses in the normal range. Patient had no complaints.

### Example No. 11

Patient Sh. Age 75. Diagnosis: Melanoma of the left lower leg, metastasis in left lymph node of the groin. In the region of the left leg swollen neoplasm of dark-brown color 5×7×9 cm in size with perifocal edema. In the region of the left groin enlarged lymph node, which was joined to the surrounding tissues. Histological diagnosis—melanoma. Treatment with a 5% ointment based on rhodamine 6G was prescribed. Over 2 months the sizes of the tumors decreased by 70%. Lymph node not determined. After 3 months, hyper-pigmentation and primary tumor determined at the site of the tumor, which was 2×3×2 cm in size. The patient did not complain of any symptoms. There was no recurrence of the melanoma over 3 years.

Thus, the Examples confirm that a readily available and inexpensive medicinal agent has been discovered, having a wide range of curative actions.

1. A method of treating a disorder selected from malignant neoplasms and amyloidoses in a subject, comprising the step of administering to said subject rhodamine 6G in a dosage ranging from 1 mg to 10 g.

2. The method of claim 1, characterized in that rhodamine 6G is used in the form of an aqueous, physiological, or alcoholic solution.

3. The method of claim 2, characterized in that an aqueous solution of rhodamine 6G is administered orally before or after meals.

4. The method of claim 1, characterized in that a solution of rhodamine 6G is administered rectally.

5. The method of claim 1, characterized in that a 1% solution of rhodamine 6G is administered intravenously.

6. The method of claim 1, characterized in that rhodamine 6G is used rectally in the form of a suppository.

7. The method of claim 1, characterized in that rhodamine 6G is used in the form of a 1-5% ointment.

8. The method of claim 1, comprising preventing the development of malignant neoplasms in a subject, comprising the step of administering to said subject rhodamine 6G in a dosage ranging from 1 mg to 10 g.
